(19) United States
(12) Patent Application Publication
(10) Pub. No.: US 2025/0331903 A1
(43) Pub. Date: Oct. 30, 2025

(54) METHODS AND DEVICES FOR BONE FIXATION

(71) Applicant: DePuy Synthes Products, Inc., Raynham, MA (US)

(72) Inventors: Richard W. CASTLE, Honey Brook, PA (US); Todd J. KENT, Philadelphia, PA (US); Veronika HEGER, Eagleville, PA (US)

(73) Assignee: DePuy Synthes Products, Inc., Raynham, MA (US)

(21) Appl. No.: 19/063,636

(22) Filed: Feb. 26, 2025

Related U.S. Application Data
(60) Provisional application No. 63/640,087, filed on Apr. 29, 2024.

(51) Int. Cl. A61B 17/84; A61B 17/04; A61B 17/86

(52) U.S. Cl. CPC A61B 17/846; A61B 17/0487; A61B 2017/8655

(57) ABSTRACT

An example apparatus is provided for bone fixation. The apparatus may include a nail, a suture lock, a tightening device, and a suture. The nail may include a proximal end and a distal end, with the suture lock being disposed at the proximal end of the nail. The suture may extend at least between the suture lock and the tightening device. Actuating the tightening device may transition the suture lock from an unlocked position to a locked position.

### CROSS-REFERENCE TO RELATED APPLICATIONS

This application claims priority under 35 U.S.C. § 119 to U.S. Provisional Application No. 63/640,087, filed Apr. 29, 2024, the entire contents of which are fully incorporated herein by reference.

### FIELD

The present disclosure generally relates to methods and devices for bone fixation. More specifically, certain embodiments relate to methods and devices for fixation of the distal tibia and distal fibula following an injury to the corresponding syndesmotic joint.

### BACKGROUND

A syndesmotic injury results when a traumatic injury damages the ligaments that span the gap between the distal tibia and fibula. This can be the result of a high ankle sprain, with no fracture of the fibula, or can also accompany a fibular fracture in a Weber B or Weber C fracture.

A surgeon can determine the presence of a syndesmotic injury by direct visualization of the joint or through radiographic imaging while positioning the ankle in a mortise view orientation. In either case, loads are applied to the joint in either a direct lateral load applied to the fibula or by applying an external rotation load to the foot. While the load is being applied, the relative distance between the fibula and the tibia, the fibula and the talus, and the tibia and the talus are observed to determine the level of damage sustained by the ligaments that typically hold the syndesmotic joint together.

If a syndesmotic injury is found to be present, the typical treatment involves stabilizing the fibula and tibia with respect to each other in the proper orientation and holding them there throughout the soft tissue healing period to allow the ligaments to re-attach and heal. In the event of a syndesmotic injury with a corresponding fibula fracture, this is done while also stabilizing the fibular fracture, which is usually accomplished with a small fracture plate on the lateral side of the fibula. Traditionally the method of stabilization has been to place one or more cortical screws across the syndesmosis, with the head against the lateral face of the fibula and the tip of the screw being in the middle of the tibia or in the medial cortex of the tibia.

This form of treatment, whereby the lateral aspect of the syndesmotic fixation sits on the lateral face of the bone, can result in unstable ankle fractures as any weight or force provided by the lateral aspect can be transferred through the fibular fracture itself. Additionally, any retained hardware under a lateral malleolar incision may cause irritation and discomfort to the skin and soft tissue.

Existing fibula nailing systems aimed at overcoming the above challenges present their own concerns in terms of adjustability in achieving appropriate targeting for syndesmotic fixation. Accordingly, alternative methods and devices for bone fixation for syndesmosis repair of the distal tibia and fibula following a syndesmotic injury would be useful.

### SUMMARY

The present disclosure is directed to apparatus and methods for stabilizing a joint between two bones, e.g., the tibia and fibula, during the soft tissue healing period following a traumatic injury.

In an exemplary application, the apparatus and methods herein may be configured to provide substantially rigid tensile fixation between the tibia and fibula while allowing the small amount of shear and rotational motion required for a standard gait. This may make it possible for patients to return to weight-bearing earlier, which may improve clinical outcomes, and/or may also reduce the number of follow-up hardware-removal surgeries.

An example apparatus is provided for bone fixation. The apparatus may include a nail, a suture lock, a tightening device, and a suture. The nail may include a proximal end and a distal end, with the suture lock being disposed at the proximal end of the nail. The suture may extend at least between the suture lock and the tightening device.

The nail may include an internal channel in which the suture may be disposed.

The tightening device when actuated may be configured to pull the suture in a distal direction within the internal channel thereby transitioning the suture lock from an unlocked position to a locked position. The tightening device may include a drive recess such that actuating the tightening device includes turning the drive recess perpendicularly to at least a portion of the nail.

The suture lock may be external to the nail in the locked and unlocked positions.

The suture lock may be disposed in the internal channel in the unlocked and locked positions such that transitioning the suture lock from the unlocked position to the locked position may force a nail wall of the nail radially outward.

The locked position of the suture lock may include a compressed state of the unlocked position. The outer diameter of the suture lock may be greater in the locked position compared to in the unlocked position. The suture lock may include a semi-rigid device configured to expand and secure itself into a bone when transitioned from the unlocked to the locked position.

The internal recess may be configured to house an end of the suture. The internal recess may surround the tightening device. The end of the suture may wrap around the tightening device in the internal recess when the suture lock is in the locked position.

The tightening device may include a locking device, wherein the suture is disposed within the internal channel and further extends at least between and beyond the locking device and the distal end of the nail. The locking device may be configured to prevent the suture from moving proximally within the internal channel thereby preventing the suture lock from transitioning from a locked position to an unlocked position.

An example method is provided for fixating an apparatus in a first bone, for example, a fibula. The method may include delivering an apparatus into a first bone, where the apparatus includes (i) a nail having a proximal end, distal end, and internal channel, (ii) a suture lock disposed at the proximal end of the nail, (iii) a tightening device, and (iv) a suture disposed within the internal channel and extending at least between the suture lock and the tightening device.

The method may further include securing the apparatus proximally within the first bone by actuating the tightening device thereby pulling the suture in a distal direction within the internal channel thereby transitioning the suture lock from an unlocked position to a locked position.

The apparatus may further include a connection, wherein delivering the apparatus into the first bone is conducted by engaging a delivery device with the connection.

The tightening device may include a drive recess, wherein actuating the tightening device includes turning the drive recess perpendicularly to at least a portion of the nail.

Transitioning the suture lock from the unlocked to the locked position may include compressing and radially expanding the suture lock.

Another example method is provided for fixating an apparatus in a first bone, for example, a fibula. The method may include delivering an apparatus into a first bone, where the apparatus includes (i) a nail having a proximal end, distal end, and internal channel, (ii) a suture lock disposed at the proximal end of the nail, (iii) a locking device, and (iv) a suture disposed within the internal channel and extending at least between the suture lock and the distal end of the nail.

The method may further include securing the apparatus proximally within the first bone by pulling the suture in a distal direction within the internal channel thereby transitioning the suture lock from an unlocked position to a locked position. In the locked position, the locking device may prevent the suture from moving proximally within the internal channel thereby preventing the suture lock from transitioning from the locked to the unlocked position. Pulling the suture in the distal direction may include pulling one or more suture distal ends beyond the distal end of the nail.

### BRIEF DESCRIPTION OF THE DRAWINGS

The above and further aspects of this disclosure are further discussed with reference to the following description in conjunction with the accompanying drawings, in which like numerals indicate like structural elements and features in various figures. The drawings are not necessarily to scale, emphasis instead being placed upon illustrating principles of the disclosure. The figures depict one or more implementations of the inventive devices, by way of example only, not by way of limitation.

FIG. 1A is an illustration of an example system for bone fixation, according to aspects of the present disclosure.

FIG. 1B is an illustration of an example apparatus used for bone fixation, according to aspects of the present disclosure.

FIG. 2 is a perspective view of an example apparatus used for bone fixation with the apparatus being in an unlocked position, according to aspects of the present disclosure.

FIG. 3 is a perspective view of an example apparatus used for bone fixation with the apparatus being in a locked position, according to aspects of the present disclosure

FIGS. 4A-4B are cross-sectional views of an example apparatus in an unlocked position (A) and locked position (B), according to aspects of the present disclosure.

FIGS. 5A-5B are cross-sectional views of an example apparatus in an unlocked position (A) and locked position (B), according to aspects of the present disclosure.

FIGS. 6A-6B are cross-sectional views of an example apparatus in an unlocked position (A) and locked position (B), according to aspects of the present disclosure.

FIG. 7 is a cross-sectional view of an example apparatus in a locked position, according to aspects of the present disclosure.

FIG. 8 is a flowchart of a method for bone fixation, according to aspects of the present disclosure.

FIG. 9 is a flowchart of a method for bone fixation, according to aspects of the present disclosure.

### DETAILED DESCRIPTION

As used herein, the terms “about” or “approximately” for any numerical values or ranges indicate a suitable dimensional tolerance that allows the part or collection of components to function for its intended purpose as described herein. More specifically, “about” or “approximately” may refer to the range of values ±20% of the recited value, e.g., “about 90%” may refer to the range of values from 71% to 99%.

The example devices and methods of treatment described herein generally involve providing syndesmosis repair of two bones, such as the tibia and fibula bones, via lateral fixation in the fibula bone. Existing fixation apparatus typically require proximal locking features be deployed early in the delivery and fixation procedure and prior to the insertion of any distal locking features (e.g., screws, suture fixation). As such, once a proximal locking feature is engaged, the screw angulation can no longer be adjusted to achieve appropriate targeting for the syndesmotic fixation. Additionally, any further reduction must be achieved by manipulating the distal bone fragment over the distal end of the apparatus. Accordingly, the devices and methods of treatment described herein overcome these challenges by providing an apparatus including a nail with an integrated suture locking device at the proximal end. The nail may be inserted retrograde through the distal end of the fibula such that distal locking features can be secured through and perpendicularly to the nail for the approximation of the fibula and tibia. The suture locking device may then be deployed through a stab incision to provide interference with the intermedullary canal, and the apparatus may subsequently be secured proximally.

Various example systems and methods are presented herein. Features from each example are combinable with other examples as understood by persons skilled in the pertinent art.

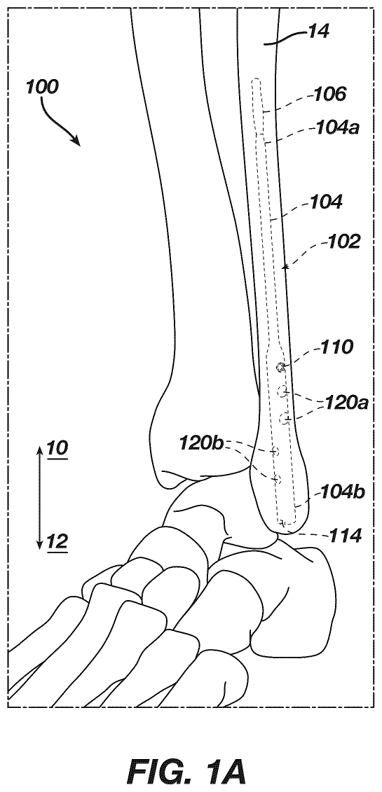

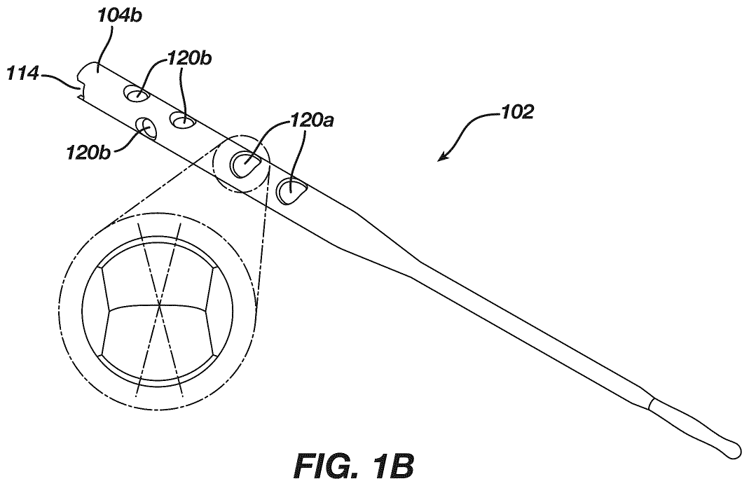

FIG. 1A is an illustration of an example system 100 for bone fixation. The system 100 may include an apparatus 102 for the approximation of two bones, such as the tibia and fibula bones. The apparatus 102 may include a nail 104, which may include a proximal end 104a and a distal end 104b, and which may be configured for insertion into a first bone 14, e.g., a fibula bone. The nail 104 may be inserted retrograde through the distal end of the fibula.

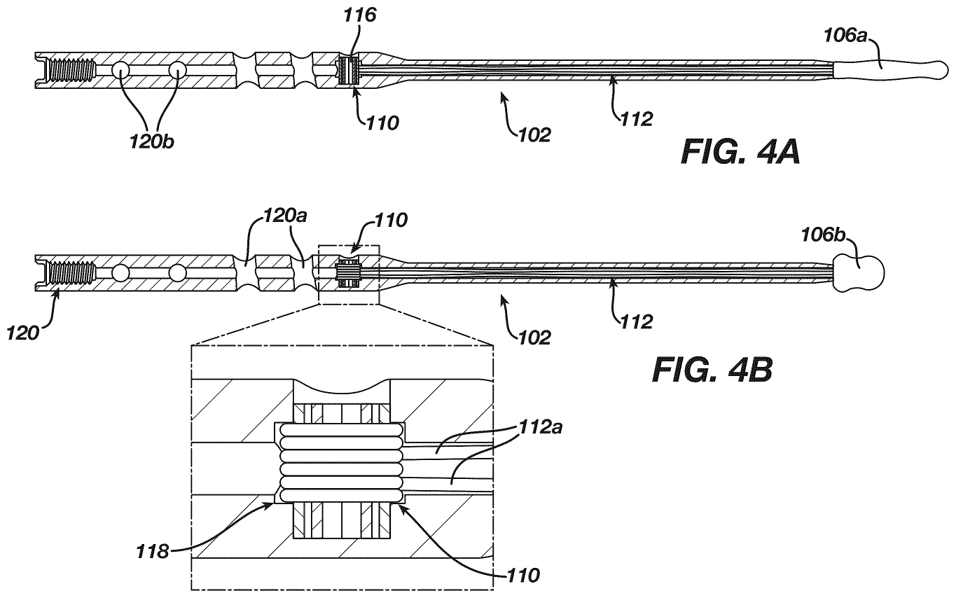

The apparatus 102 may further include a suture lock 106 disposed at the proximal end 104a of the nail 104. Suture lock 106 may be positioned in an unlocked position or locked position, as further discussed below. The apparatus 102 may further include a tightening device 110, and a suture 112 (e.g., as shown in FIGS. 4A-4B, 5A-5B, 6A-6B, 7) extending at least between the suture lock 106 and the tightening device 110.

The apparatus 102 may further include one or more fixation holes 120a configured such that one or more syndesmosis fixation features (e.g., screws) may be inserted therethrough. The apparatus 102 may further include one or more fixation holes 120b configured such that one or more distal fixation features may be inserted therethrough. As shown in FIG. 1A, fixation hole(s) 120a and 120b may be disposed perpendicularly with respect to one another and through an axis of the nail 104. The apparatus may further include an internal channel 114 through which a suture 112 may be disposed, as further discussed below. The apparatus may further include a connection 120 (e.g., as shown in FIG. 4A) configured such that engaging a delivery device with the connection 120 aids in delivering the apparatus 102 into the first bone 14.

FIG. 1B provides a perspective view of an example apparatus 102. As shown, in some embodiments, one or more fixation holes 120a may include an inner screw hole or channel having a variable angle window. Such angulation may be defined by the width of the screw channel combined with the trajectory of the channel axis, and may be configured of any angle (e.g., between 0 degrees to 30 degrees) depending on the configuration or orientation of syndesmotic fixation needed. A benefit of such feature is that after the nail 104 has been locked in place in the fibula bone via the suture lock 106, as discussed herein, the fibula and tibia bones may not be aligned in a substantially straight manner. That is, a fixation feature (e.g., screw) may need to be inserted into one or more of the fixation holes 120a at an angle to properly fixate the fibula and tibia bones with the syndesmosis. As such, an angled screw hole can allow a screw to be driven at an angle through the fibula bone, via the nail 104, and into the tibia bone in order to ensure appropriate alignment with the trans-syndesmotic angle, thus aligning the screw with the syndesmosis, even if the nail 104 has already been locked in position at some other orientation that itself may not align with the syndesmosis. While FIG. 1B illustrates the fixation hole(s) 120a as having an angled screw hole, it should be understood that any of the fixation hole(s) of the nail 104 (e.g., fixation hole(s) 120a, 120b) could be configured to have such angulation as well.

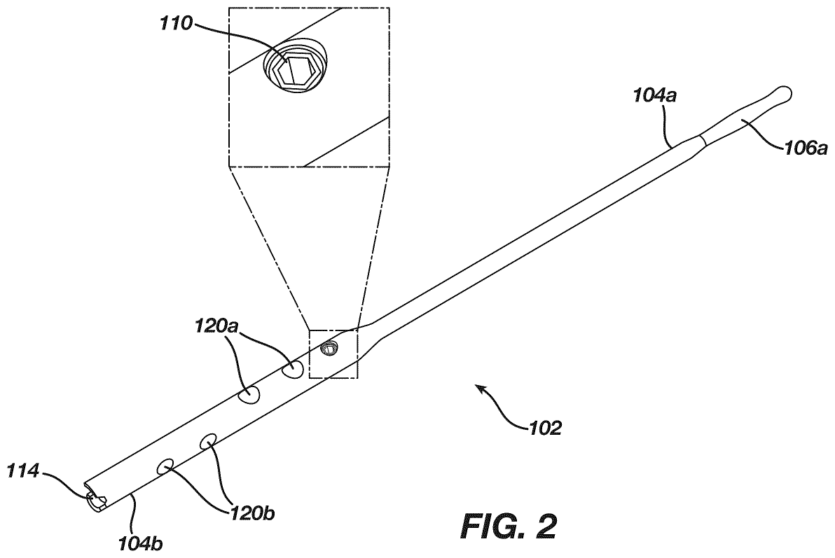

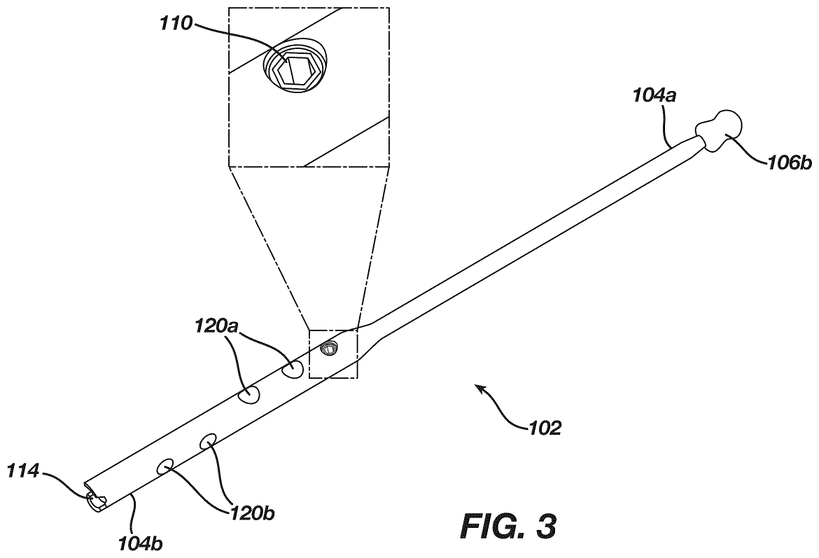

FIGS. 2 and 3 provide perspective views of an example apparatus 102 with the suture lock 106 in an unlocked position 106a (FIG. 2) and a locked position 106b (FIG. 3). The apparatus 102 may be inserted into the first bone with the suture lock 106 in the unlocked position 106a. Once the apparatus 102 is positioned within the first bone, the suture lock 106 may be transitioned from the unlocked position 106a to the locked position 106, fixing the apparatus 102 in place, as further discussed below. As shown in FIGS. 2 and 3, and discussed further below, the tightening device 110 may include a drive recess 116 (e.g., as shown in FIG. 4A), where actuating the tightening device 110 includes turning the drive recess 116 (e.g., a hex drive) perpendicularly to at least a portion of the nail 104.

FIGS. 4A and 4B provide cross-sectional views of an apparatus 102 with the suture lock 106 being in an unlocked position 106a (FIG. 4A) and a locked position 106b (FIG. 4B). The suture 112, including one or more suture threads 112a, is disposed within the internal channel 114 of the nail 104, and extends from the suture lock 106 to the tightening device 110. The internal channel 112 includes an internal recess 118 that surrounds the tightening device 110 and is configured to house an end of the suture 112.

After the apparatus 102 has been positioned within the first bone (e.g., the fibula bone), as discussed herein, the suture lock 106 can be transitioned from its unlocked position 106 to its locked position 106b, to fix the apparatus 102 in place, by actuating the tightening device 110, such as by turning the drive recess 116 perpendicularly to at least a portion of the nail 104. As the tightening device 110 is actuated, the suture 112 is pulled in a distal direction 12 within the internal channel 114, wrapping around the tightening device 110 in the internal recess 118, thereby transitioning the suture lock 106 from its unlocked position 106a to its locked position 106b. FIGS. 4B, 5B, and 6B show an example of how the suture 112 may be wrapped around tightening device 110 in the internal recess 118. As shown in FIGS. 4A and 4B, the suture lock 106 remains external to the nail 104 in both its unlocked 106a and locked 106b positions. Further, the suture lock 106 is in a compressed state and has a greater diameter when in its locked position 106b compared to its unlocked position 106a.

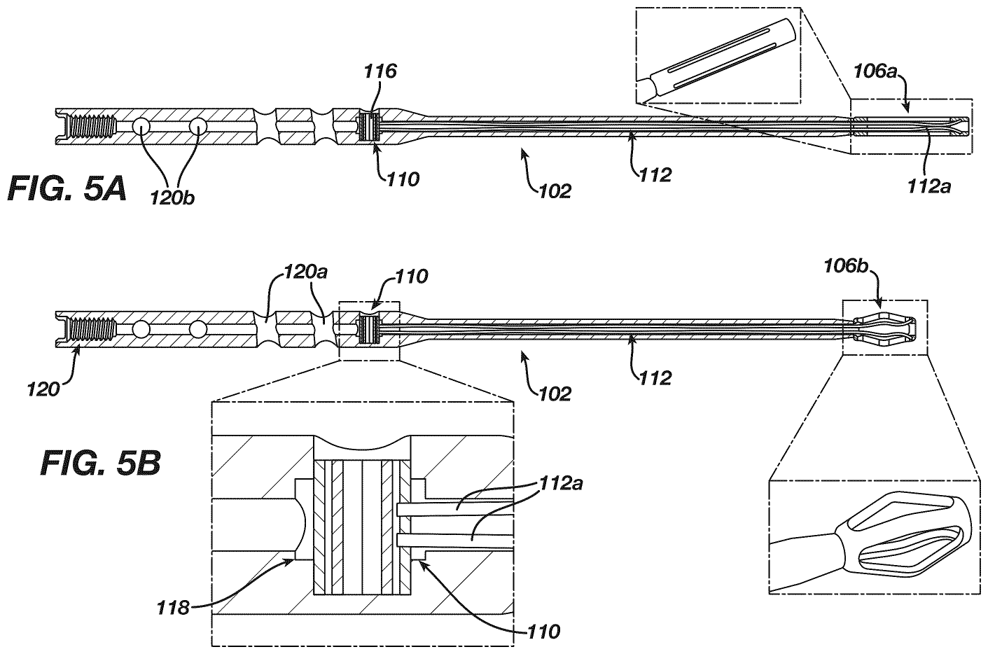

FIGS. 5A and 5B provide cross-sectional views of an apparatus 102 with the suture lock 106 being in an unlocked position 106a (FIG. 5A) and a locked position 106b (FIG. 5B). The apparatus 102 of FIGS. 5A and 5B may be similar to that of FIGS. 4A and 4B, but for the type of suture lock 106. As shown in FIGS. 5A and 5B, the suture lock 106 comprises a semi-rigid device configured to expand when it transitions from its unlocked position 106a to its locked position 106b. In expanding in such fashion, the suture lock 106 of FIGS. 5A and 5B is configured to secure itself into a bone (e.g., the fibula bone) once transitioned to its compressed and radially expanded state in its locked position 106b.

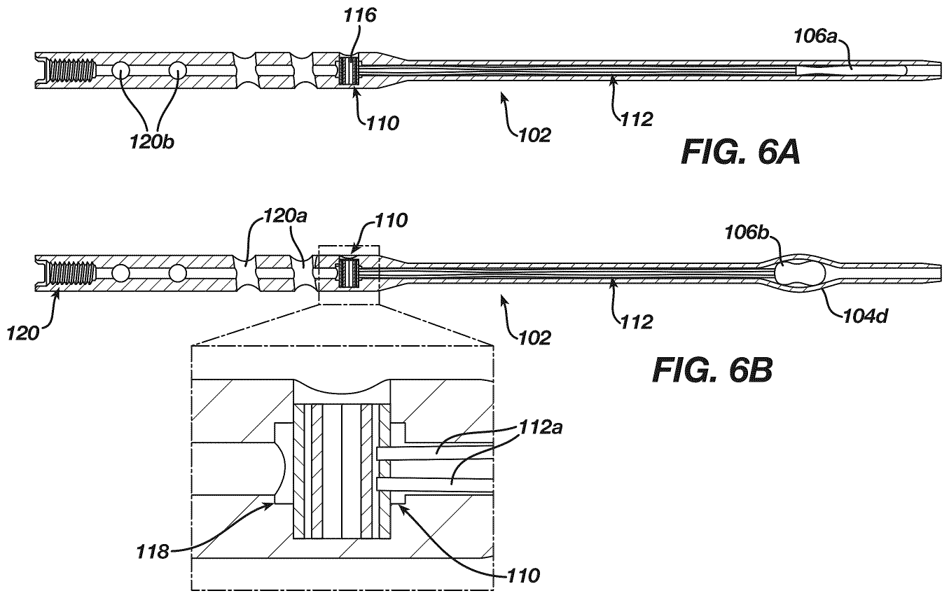

FIGS. 6A and 6B provide cross-sectional views of an apparatus 102 with the suture lock 106 being in an unlocked position 106a (FIG. 6A) and a locked position 106b (FIG. 6B). The apparatus 102 of FIGS. 6A and 6B may be similar to that of FIGS. 4A-4B and 5A-5B, but for the type of suture lock 106. As shown in FIGS. 6A and 6B, the suture lock 106 is disposed within the internal channel 114 of the nail 104 in both its unlocked position 106a and locked position 106b. As the suture lock 106 transitions from its unlocked 106a to locked 106b positions, the suture lock 106 is compressed and radially expands, forcing an external nail wall 104d of the nail 104 radially outward, thereby fixing the apparatus 102 in place within the bone.

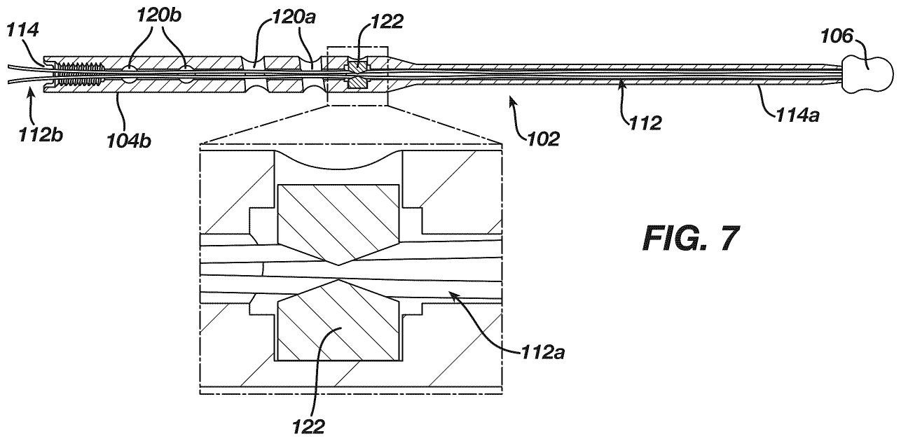

FIG. 7 provides a cross-sectional view of an apparatus 102 with the suture lock 106 in its locked position. Rather than have a similar tightening device 110, as with the apparatus 102 of FIGS. 4A-4B, 5A-5B, and 6A-6B, the apparatus 102 of FIG. 7 includes a locking device 122. As shown in FIG. 7, the suture 112 is disposed within the internal channel 114 of the nail 104, and extends not only from the suture lock 106 to the locking device 122, but also beyond the locking device 122 and the distal end 104b of the nail 104. In such embodiment, the locking device 122 is configured to prevent the suture 112 from moving proximally 10 within the internal channel 114, thereby preventing the suture lock 106 from transitioning from its locked position 106b back to its unlocked position 106a. In this way, the locking device 122 acts as a form of check valve, allowing the suture 112 to be pulled through the locking device 122 in a distal direction 12, but not in a proximal direction 10.

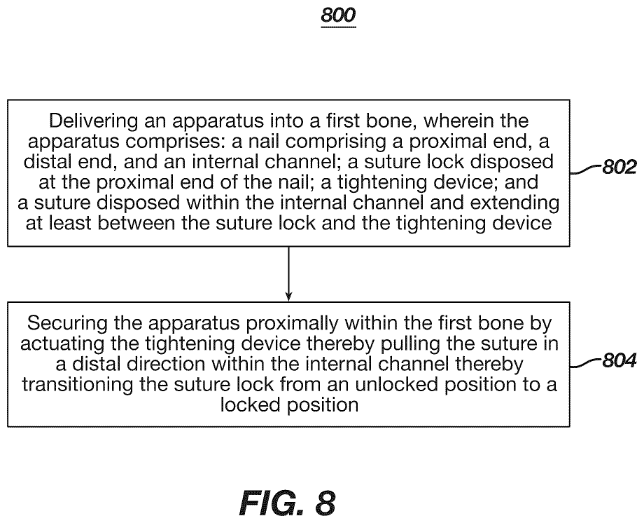

FIG. 8 provides a flowchart of a method for bone fixation. In block 802, the method may include delivering an apparatus into a first bone, such as the fibula bone. The apparatus may be the apparatus 102 as shown and described in FIGS. 4A-4B, 5A-5B, or 6A-6B. The apparatus may include a nail (e.g., nail 104) having a proximal end, a distal end, and an internal channel; a suture lock (e.g., suture lock 106) disposed at the proximal end of the nail; a tightening device (e.g., tightening device 110); and a suture (e.g., suture 112) disposed within the internal channel and extending at least between the suture lock and the tightening device. In block 804, the method may include securing the apparatus proximally within the first bone by actuating the tightening device thereby pulling the suture in a distal direction within the internal channel thereby transitioning the suture lock from an unlocked position to a locked position.

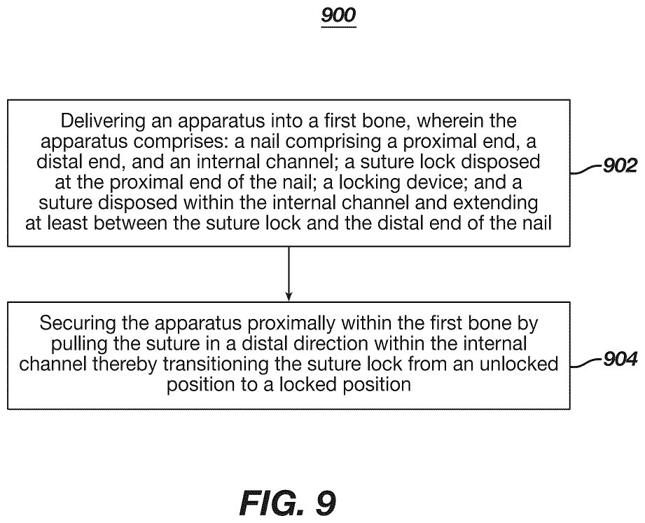

FIG. 9 provides a flowchart of a method for bone fixation. In block 902, the method may include delivering an apparatus into a first bone, such as the fibula bone. The apparatus may be the apparatus 102 as shown and described in FIG. 7. The apparatus may include a nail (e.g., nail 104) having a proximal end, a distal end, and an internal channel; a suture lock (e.g., suture lock 106) disposed at the proximal end of the nail; a locking device (e.g., locking device 122); and a suture (e.g., suture 112) disposed within the internal channel and extending at least between the suture lock and the distal end of the nail. In block 904, the method may include securing the apparatus proximally within the first bone by pulling the suture in a distal direction within the internal channel thereby transitioning the suture lock from an unlocked position to a locked position. Pulling the suture in the distal direction may include pulling one or more suture distal ends beyond the distal end of the nail.

The descriptions contained herein are examples of embodiments of the disclosure and are not intended in any way to limit the scope of the disclosure. As described herein, the disclosure contemplates many variations and modifications of structures and methods, including alternative materials, alternative configurations of component parts, and alternative method steps. Modifications and variations apparent to those having skill in the pertinent art according to the teachings of this disclosure are intended to be within the scope of the claims which follow.

The disclosed technology described herein can be further understood according to the following clauses.

Clause 1: An apparatus for bone fixation, comprising: a nail comprising a proximal end and a distal end; a suture lock disposed at the proximal end of the nail; a tightening device; and a suture extending at least between the suture lock and the tightening device.

Clause 2: The apparatus of clause 1, wherein the nail comprises an internal channel, and wherein the suture is disposed within the internal channel.

Clause 3: The apparatus of clause 2, wherein the tightening device when actuated is configured to pull the suture in a distal direction within the internal channel thereby transitioning the suture lock from an unlocked position to a locked position.

Clause 4: The apparatus of clause 3, wherein the tightening device comprises a drive recess, and wherein actuating the tightening device comprises turning the drive recess perpendicularly to at least a portion of the nail.

Clause 5: The apparatus of clause 3, wherein the suture lock is external to the nail in the unlocked and locked positions.

Clause 6: The apparatus of clause 3, wherein the suture lock is disposed within the internal channel in the unlocked and locked positions, and wherein transitioning the suture lock from the unlocked position to the locked position forces a nail wall of the nail radially outward.

Clause 7: The apparatus of clause 3, wherein the locked position of the suture lock comprises a compressed state of the unlocked position.

Clause 8: The apparatus of clause 3, wherein an outer diameter of the suture lock is greater in the locked position compared to the unlocked position.

Clause 9: The apparatus of clause 3, wherein the suture lock comprises a semi-rigid device configured to expand and secure itself into a bone when transitioned from the unlocked position to the locked position.

Clause 10: The apparatus of clause 3, wherein the internal channel has an internal recess configured to house an end of the suture, and wherein the internal recess surrounds the tightening device.

Clause 11: The apparatus of clause 10, wherein the end of the suture wraps around the tightening device in the internal recess when the suture lock is in the locked position.

Clause 12: The apparatus of clause 2, wherein the tightening device comprises a locking device, and wherein the suture is disposed within the internal channel and further extends at least between and beyond the locking device and the distal end of the nail.

Clause 13: The apparatus of clause 12, wherein the locking device is configured to prevent the suture from moving proximally within the internal channel thereby preventing the suture lock from transitioning from a locked position to an unlocked position.

Clause 14: A method for bone fixation, the method comprising: delivering an apparatus into a first bone, wherein the apparatus comprises: a nail comprising a proximal end, a distal end, and an internal channel; a suture lock disposed at the proximal end of the nail; a tightening device; and a suture disposed within the internal channel and extending at least between the suture lock and the tightening device; and securing the apparatus proximally within the first bone by actuating the tightening device thereby pulling the suture in a distal direction within the internal channel thereby transitioning the suture lock from an unlocked position to a locked position.

Clause 15: The method of clause 14, wherein the apparatus further comprises a connection, and wherein delivering the apparatus into the first bone is conducted by engaging a delivery device with the connection.

Clause 16: The method of clause 14, wherein the tightening device comprises a drive recess, and wherein actuating the tightening device comprises turning the drive recess perpendicularly to at least a portion of the nail.

Clause 17: The method of clause 14, wherein transitioning the suture lock from the unlocked position to the locked position comprises compressing and radially expanding the suture lock.

Clause 18: A method for bone fixation, the method comprising: delivering an apparatus into a first bone, wherein the apparatus comprises: a nail comprising a proximal end, a distal end, and an internal channel; a suture lock disposed at the proximal end of the nail; a locking device; and a suture disposed within the internal channel and extending at least between the suture lock and the distal end of the nail; and securing the apparatus proximally within the first bone by pulling the suture in a distal direction within the internal channel thereby transitioning the suture lock from an unlocked position to a locked position.

Clause 19: The method of clause 18, wherein, in the locked position, the locking device prevents the suture from moving proximally within the internal channel thereby preventing the suture lock from transitioning from the locked position to the unlocked position.

Clause 20: The method of clause 18, wherein pulling the suture in the distal direction comprises pulling one or more suture distal ends beyond the distal end of the nail.

What is claimed is:

1. An apparatus for bone fixation, comprising:
a nail comprising a proximal end and a distal end;
a suture lock disposed at the proximal end of the nail;
a tightening device; and
a suture extending at least between the suture lock and the tightening device.

2. The apparatus of claim 1, wherein the nail comprises an internal channel, and wherein the suture is disposed within the internal channel.

3. The apparatus of claim 2, wherein the tightening device when actuated is configured to pull the suture in a distal direction within the internal channel thereby transitioning the suture lock from an unlocked position to a locked position.

4. The apparatus of claim 3, wherein the tightening device comprises a drive recess, and wherein actuating the tightening device comprises turning the drive recess perpendicularly to at least a portion of the nail.

5. The apparatus of claim 3, wherein the suture lock is external to the nail in the unlocked and locked positions.

6. The apparatus of claim 3, wherein the suture lock is disposed within the internal channel in the unlocked and locked positions, and wherein transitioning the suture lock from the unlocked position to the locked position forces a nail wall of the nail radially outward.

7. The apparatus of claim 3, wherein the locked position of the suture lock comprises a compressed state of the unlocked position.

8. The apparatus of claim 3, wherein an outer diameter of the suture lock is greater in the locked position compared to the unlocked position.

9. The apparatus of claim 3, wherein the suture lock comprises a semi-rigid device configured to expand and secure itself into a bone when transitioned from the unlocked position to the locked position.

10. The apparatus of claim 3, wherein the internal channel has an internal recess configured to house an end of the suture, and wherein the internal recess surrounds the tightening device.

11. The apparatus of claim 10, wherein the end of the suture wraps around the tightening device in the internal recess when the suture lock is in the locked position.

12. The apparatus of claim 2, wherein the tightening device comprises a locking device, and wherein the suture is disposed within the internal channel and further extends at least between and beyond the locking device and the distal end of the nail.

13. The apparatus of claim 12, wherein the locking device is configured to prevent the suture from moving proximally within the internal channel thereby preventing the suture lock from transitioning from a locked position to an unlocked position.

14. A method for bone fixation, the method comprising:
delivering an apparatus into a first bone, wherein the apparatus comprises:
a nail comprising a proximal end, a distal end, and an internal channel;
a suture lock disposed at the proximal end of the nail;
a tightening device; and
a suture disposed within the internal channel and extending at least between the suture lock and the tightening device; and
securing the apparatus proximally within the first bone by actuating the tightening device thereby pulling the suture in a distal direction within the internal channel thereby transitioning the suture lock from an unlocked position to a locked position.

15. The method of claim 14, wherein the apparatus further comprises a connection, and wherein delivering the apparatus into the first bone is conducted by engaging a delivery device with the connection.

16. The method of claim 14, wherein the tightening device comprises a drive recess, and wherein actuating the tightening device comprises turning the drive recess perpendicularly to at least a portion of the nail.

17. The method of claim 14, wherein transitioning the suture lock from the unlocked position to the locked position comprises compressing and radially expanding the suture lock.

18. A method for bone fixation, the method comprising:
delivering an apparatus into a first bone, wherein the apparatus comprises:
a nail comprising a proximal end, a distal end, and an internal channel;
a suture lock disposed at the proximal end of the nail;
a locking device; and
a suture disposed within the internal channel and extending at least between the suture lock and the distal end of the nail; and
securing the apparatus proximally within the first bone by pulling the suture in a distal direction within the internal channel thereby transitioning the suture lock from an unlocked position to a locked position.

19. The method of claim 18, wherein, in the locked position, the locking device prevents the suture from moving proximally within the internal channel thereby preventing the suture lock from transitioning from the locked position to the unlocked position.

20. The method of claim 18, wherein pulling the suture in the distal direction comprises pulling one or more suture distal ends beyond the distal end of the nail.
